(19) United States
(12) Patent Application Publication
(10) Pub. No.: US 2025/0082286 A1
(43) Pub. Date: Mar. 13, 2025

(54) BEARING DEVICE AND MOBILE DIGITAL RADIOGRAPHY DEVICE

(71) Applicant: SHANGHAI UNITED IMAGING HEALTHCARE CO., LTD., Shanghai (CN)

(72) Inventor: Lei LIU, Shanghai (CN)

(73) Assignee: SHANGHAI UNITED IMAGING HEALTHCARE CO., LTD., Shanghai (CN)

(21) Appl. No.: 18/955,942

(22) Filed: Nov. 21, 2024

Related U.S. Application Data
(63) Continuation of application No. PCT/CN2023/100307, filed on Jun. 14, 2023.

(30) Foreign Application Priority Data
Jun. 14, 2022 (CN) 202210674602.X

(51) Int. Cl. A61B 6/00

(52) U.S. Cl. CPC A61B 6/4405; A61B 6/4429

(57) ABSTRACT

A bearing device and a mobile digital radiography device are provided. The Bearing device may include a mount, a support unit, a vibration absorption unit, and a connecting member. The mount may be connected to the support unit. The support unit may be connected to the connecting member through the vibration absorption unit. The vibration absorption unit may include a first elastic member and a second elastic member. The first elastic member and the second elastic member may be disposed in different directions.

### CROSS-REFERENCE TO RELATED APPLICATIONS

This application is a continuation of International Application No. PCT/CN2023/100307, filed on Jun. 14, 2023, which claims priority of Chinese Patent Application No. 202210674602.X, filed on Jun. 14, 2022, the entire contents of each of which are hereby incorporated by reference.

### TECHNICAL FIELD

The present disclosure relates to the technical field of a medical device, and in particular, to a bearing device and a mobile digital radiography device.

### BACKGROUND

A mobile digital radiography (DR) device is an advanced medical device formed by combining a computer digital image processing technology and an X-ray radiation technology. The mobile digital radiography device is widely used in clinical practice because of low radiation dose, high image quality, high disease detection rate, and diagnostic accuracy during shooting.

An X-ray tube may generate X-rays for generating fluoroscopic images, and is an important part of the mobile digital radiography device. Since a weight of the X-ray tube and the bearing device is relatively large and overall rigidity of the mobile digital radiography device is relatively large, vibration generated by movement (e.g., lifting movement) of the mobile digital radiography device may bring a certain impact to the X-ray tube, which is easy to damage the X-ray tube and reduces the service life and safety.

Therefore, it is desirable to provide a bearing device to effectively alleviate the problem of excessive vibration amplitude of the X-ray tube.

### SUMMARY

One or more embodiments of the present disclosure provide a bearing device. The bearing device may include a mount, a support unit, and a vibration absorption unit. The mount may be connected to the support unit. The support unit may be connected to the vibration absorption unit. The vibration absorption unit may include at least one elastic member.

One or more embodiments of the present disclosure provide a mobile digital radiography device. The mobile digital radiography device may include an X-ray tube, a beam limiter, and the bearing device. The X-ray tube may be disposed on one side of the mount. A hole may be disposed at a position corresponding to the X-ray tube of the mount for a beam of the X-ray tube to pass through. The beam limiter disposed on the other side of the mount may be configured to receive the beam passing through the hole.

One or more embodiments of the present disclosure provide a bearing device of an X-ray tube. The bearing device of an X-ray tube may include a bottom plate, a support unit, and a vibration absorption unit. The support unit may be disposed on the bottom plate and may be disposed close to the X-ray tube. The vibration absorption unit may be disposed on the support unit and include a first elastic member and a second elastic member. A direction where the first elastic member is disposed may be parallel to a plane where the support unit is located. An inclination angle may be formed between the direction where the first elastic member is disposed and a plane where the bottom plate is located. A direction where the second elastic member is disposed may be perpendicular to the plane where the support unit is located.

One or more embodiments of the present disclosure provide a mobile digital radiography imaging device. The mobile digital radiography imaging device may include the bearing device of the X-ray tube and the X-ray tube. The X-ray tube may be installed on the bearing device of the X-ray tube.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which the same reference numbers represent the same structures, wherein:

FIG. 1 is a schematic diagram illustrating a structure of an exemplary bearing device according to some embodiments of the present disclosure;

FIG. 2 is a schematic diagram illustrating a structure of an exemplary bearing device according to other embodiments of the present disclosure;

FIG. 3 is a schematic diagram illustrating an exemplary structure of connection between a second connecting member and a second vibration absorption component, and an exemplary structure of connection between a second support component and the second vibration absorption component according to some embodiments of the present disclosure;

FIG. 4 is a schematic diagram illustrating an exemplary structure of connection between a first connecting member and a first vibration absorption component, and an exemplary structure of connection between a first support component and the first vibration absorption component according to some embodiments of the present disclosure;

FIG. 5 is a schematic diagram illustrating an exemplary structure of connection between a first connecting member and a first support shaft according to some embodiments of the present disclosure;

FIG. 6 is a partial cross-sectional view of an A-A′ direction in FIG. 2;

FIG. 7 is a schematic diagram illustrating an exemplary structure of connection between a bearing device and an X-ray tube according to some embodiments of the present disclosure;

FIG. 8 is a schematic diagram illustrating an exemplary structure of connection between a bearing device and a beam limiter, and an exemplary structure of connection between the bearing device and an X-ray tube according to some embodiments of the present disclosure;

FIG. 9 is a schematic diagram illustrating an exemplary structure of connection between a bearing device and a telescopic arm according to some embodiments of the present disclosure; and

FIG. 10 is a schematic diagram illustrating an exemplary structure of a mobile digital radiography device according to some embodiments of the present disclosure.

### DETAILED DESCRIPTION

In order to more clearly illustrate the technical solutions related to the embodiments of the present disclosure, a brief introduction of the drawings referred to the description of the embodiments is provided below. Obviously, drawings described below are only some examples or embodiments of the present disclosure. Those skilled in the art, without further creative efforts, may apply the present disclosure to other similar scenarios according to these drawings. Unless obviously obtained from the context or the context illustrates otherwise, the same numeral in the drawings refers to the same structure or operation.

As used in the disclosure and the appended claims, the singular forms “a,” “an,” and “the” include plural referents unless the content clearly dictates otherwise; the plural forms may be intended to include singular forms as well. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

An X-ray tube may generate X-rays for generating a fluoroscopic image, and is an important part of a mobile digital radiography device. In some embodiments, the X-ray tube may be usually fixed on a base of the mobile digital radiography device, and the base may be connected to a support frame of the mobile digital radiography device. The X-ray tube may be connected to a moving device (e.g., a mobile base, a C-arm, or a telescopic arm) of the mobile digital radiography device, and the X-ray tube may be driven to move through the moving device. For example, the X-ray tube may be driven to move up and down, or the X-ray tube may be driven to move horizontally. However, at present, most X-ray tubes may be fixed to the base by means of hoops, and connected to the support frame and the moving device of the mobile digital radiography device through bolts. Since a weight of the X-ray tube and its bearing device is relatively large and overall rigidity of the mobile digital radiography device is relatively large, vibration generated by movement (e.g., lifting movement) of the mobile digital radiography device may bring a certain impact to the X-ray tube, and in particular, vibration generated when moving over a threshold may be transmitted to the X-ray tube to form a relatively large impact, which may be easy to damage the X-ray tube. For example, the impact may damage a bearing used to fix a rotating anode in the X-ray tube, thereby causing the rotating anode of the X-ray tube to fall off and affecting the safety of use of the X-ray tube.

In some embodiments, in order to solve the problem that the X-ray tube is easily damaged, a structure of a tower wheel and a balancer may be used for vibration absorption, e.g., the X-ray tube may be connected to the balancer through a wire rope (wound on the tower wheel), and when the mobile digital radiography device moves (e.g., lifting movement), a spring on the balancer may expand and contract to absorb vibration energy, and a moment balance of the X-ray tube at any position within a movable range may be maintained, thereby achieving the vibration absorption. However, in the actual application process, a position of the wire rope connecting the X-ray tube and the balancer may change along an axial direction of the tower wheel, so that the wire rope may not always remain perpendicular to a horizontal plane, which may lead to an increase in frictional resistance between the wire rope and a rope groove of the tower wheel, and reduce the reliability and service life of the wire rope. In some embodiments, a rotating shaft of the tower wheel may be replaced with a lead screw, the tower wheel may be fixedly connected with a nut, and the tower wheel may move together along the axial direction during the lifting movement, thereby avoiding the position of the wire rope from changing along the axial direction of the tower wheel. However, a cooperation structure of a lead screw pair and the tower wheel may be relatively complicated, which may make the structure of the mobile digital radiography device more complicated, and the costs of the lead screw pair may be relatively high, which may increase the costs of the mobile digital radiography device to a certain extent. In some other embodiments, friction sheets may be added at joints between the X-ray tube and two ends of the support frame to balance a position of the X-ray tube, thereby playing a certain vibration absorption effect. However, the solution of adding the friction plates may merely be suitable for fine-tuning the position of the X-ray tube, which may improve a projection effect of a beam emitted by the X-ray tube and may not achieve a good vibration absorption effect for relatively strong impacts.

According to the above reasons, the present disclosure provides a bearing device. The bearing device may be used to bear the X-ray tube and connect the X-ray tube to the moving device (e.g., the C-arm, the mobile base, or the telescopic arm) of the mobile digital radiography device. When the moving device of the mobile digital radiography device moves, the vibration generated by the movement of the mobile digital radiography device may be buffered in a plurality of dimensions by a vibration absorption unit disposed on a bearing unit, thereby reducing the impact on the X-ray tube, avoiding damage to the X-ray tube, and improving the stability, safety and service life of the X-ray tube.

It should be noted that the description of the application scenario of the bearing device in the present disclosure is provided merely for the purpose of illustration, intended to describe an exemplary application scenario of the bearing device, and does not limit the use of the bearing device in a mobile digital radiography device. For example, the bearing device may be used to bear a linear accelerator. As another example, the bearing device may be used to bear a detector of a radiography imaging device, so as to reduce the vibration transmitted to the detector by a gantry of the radiography imaging device.

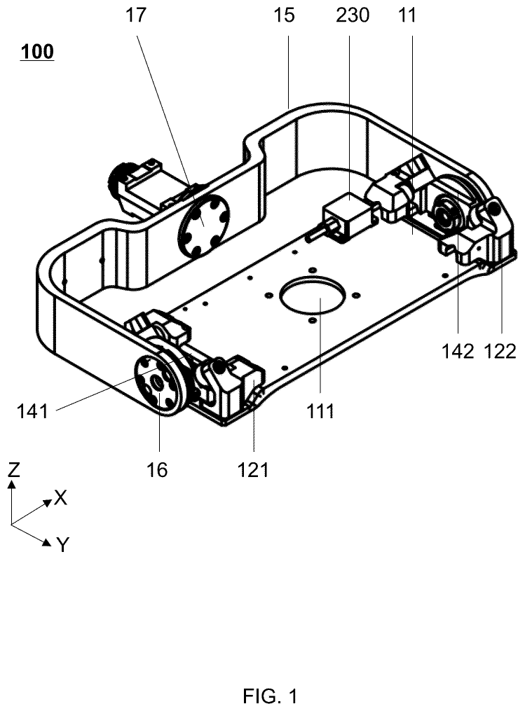

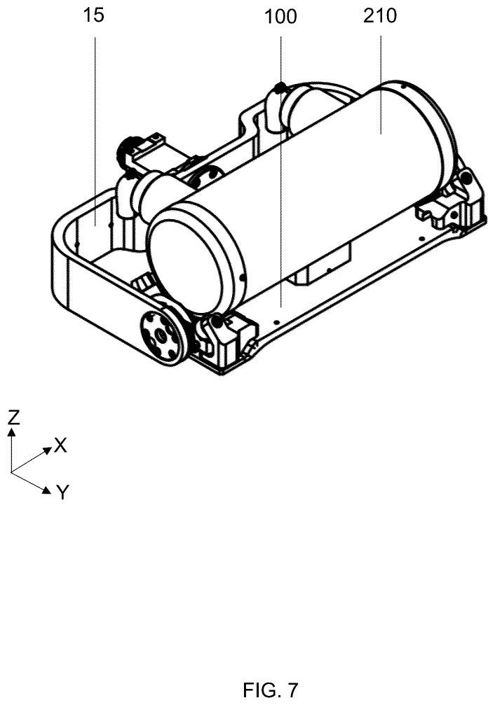

In some embodiments, as shown in FIGS. 1-5, a bearing device 100 may include a mount 11, a support unit 12, and a vibration absorption unit 13. The mount 11 may be connected to the support unit 12. The support unit 12 may be connected to the vibration absorption unit 13. The vibration absorption unit 13 may include at least one elastic member. The mount 11 may be used to bear a specific object (e.g., an X-ray tube 210 of a mobile digital radiography device 200 in FIG. 7). The support unit 12 may be used to connect the vibration absorption unit 13 to the mount 11. The at least one elastic member of the vibration absorption unit 13 may absorb and buffer external impact force using elasticity thereof, so that vibration may be buffered and absorbed when transmitted to the support unit 12 and the mount 11, thereby improving stability and safety of use of the specific object disposed on the mount 11.

In some cases, the at least one elastic member absorbs and buffers the vibration, which can prolong service life of the X-ray tube 210, and improve safety of use of the mobile digital radiography device.

In some embodiments, the vibration absorption unit 13 may include a first elastic member 131 and a second elastic member 132. The first elastic member 131 and the second elastic member 132 may be disposed in different directions. Directions where the first elastic member 131 and the first elastic member 132 are disposed refer to axial placement directions of the first elastic member 131 and the first elastic member 132. The vibration transmitted by the connecting member 14 may be decomposed into components in three directions, namely the component parallel to a length direction (which may be indicated by the arrow X in FIG. 1) of the mount 11, the component parallel to a width direction (which may be indicated by the arrow Y in FIG. 1) of the mount 11, and the component in a height direction (which may be indicated by the arrow Z in FIG. 1) parallel to the mount 11.

In some cases, since the first elastic member 131 and the second elastic member 132 are disposed in different directions, the first elastic member 131 and the second elastic member 132 may absorb and buffer the vibration respectively in different directions (e.g., an extension direction of the first elastic member 131 and an extension direction of the second elastic member 132). In addition, since the vibration may be decomposed into the components in the three directions, the first elastic member 131 and the second elastic member 132 may be disposed to absorb and buffer the vibration in different directions, which can synchronously perform vibration absorption on a plurality of degrees of freedom of the X-ray tube 210, realize dynamic balance of the X-ray tube 210, prolong service life of the X-ray tube 210, and improve safety of use of the mobile digital radiography device.

In some embodiments, the direction where the first elastic member 131 is disposed may be perpendicular to the direction where the second elastic member 132 is disposed. Merely by way of example, the direction where the first elastic member 131 is disposed may be parallel to the width direction of the mount 11, and the direction where the second elastic member 132 is disposed may be parallel to the length direction of the mount 11. At this time, the first elastic member 131 may absorb and buffer the vibration in the Y direction, and the second elastic member 132 may absorb and buffer the vibration in the X direction, so the vibration absorption unit 13 may simultaneously buffer the vibration in the X direction and the Y direction. As another example, the direction where the first elastic member 131 is disposed may be parallel to the height direction of the mount 11, and the direction where the second elastic member 132 is disposed may be parallel to the width direction of the mount 11. At this time, the first elastic member 131 may absorb and buffer the vibration in the Z direction, and the second elastic member 132 may absorb and buffer the vibration in the Y direction, so the vibration absorption unit 13 may simultaneously buffer the vibration in the Z direction and the Y direction.

In some embodiments, the direction where the first elastic member 131 is disposed may not be perpendicular to the direction where the second elastic member 132 is disposed. Merely by way of example, the direction where the first elastic member 131 is disposed may be parallel to the length direction of the mount 11, so the first elastic member 131 may buffer the vibration in the X direction. The second elastic member 132 may be located in an X-Y plane, and an angle of 30° may be formed between the direction where the second elastic member 132 is disposed and the width direction of the mount 11, so the second elastic member 132 may simultaneously buffer the vibration in the X direction and the Y direction. As another example, the direction where the first elastic member 131 is disposed may be parallel to the height direction of the mount 11, so the first elastic member 131 may buffer the vibration in the Z direction. The second elastic member 132 may be located in an X-Z plane, and an angle of 45° may be formed between the direction where the second elastic member 132 is disposed and the length direction of the mount 11, so the second elastic member 132 may buffer the vibration in the X direction and the Z direction.

In some embodiments, the first elastic member 131 and the second elastic member 132 may be coplanarly disposed. For example, the direction where the first elastic member 131 is disposed may be parallel to the height direction of the mount 11, and the second elastic member 132 may be located in a same plane as the first elastic member 131, and an angle of non-90° may be formed between the second elastic member 132 and the length direction of the mount 11.

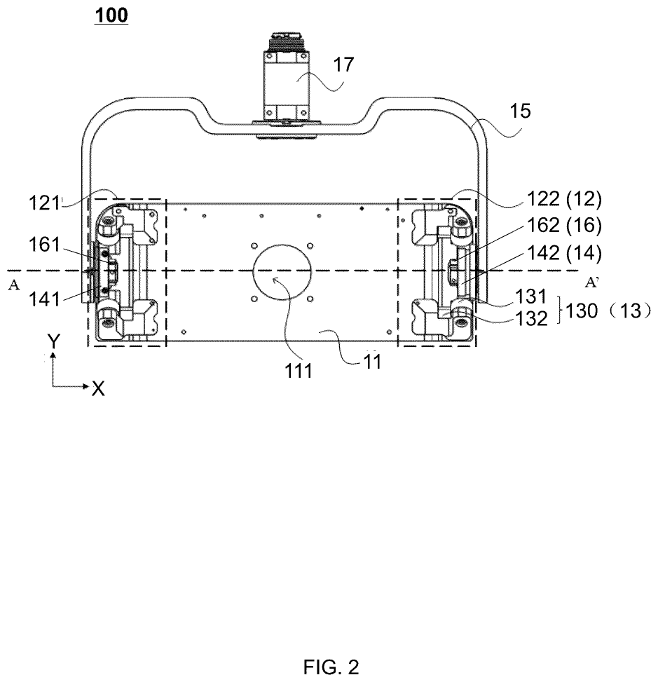

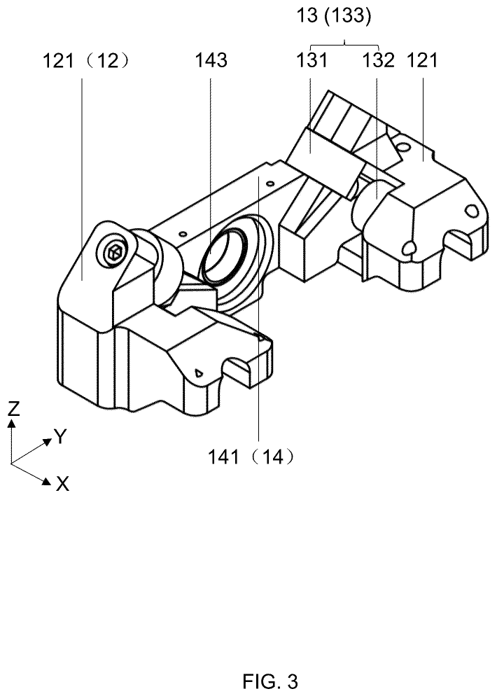

In some embodiments, the direction where the first elastic member 131 is disposed may intersect a plane where the mount 11 is located, and the direction where the second elastic member 132 is disposed may be parallel to the plane where the mount 11 is located. Merely by way of example, as shown in FIGS. 2-3, the direction where the first elastic member 131 is disposed may intersect the plane where the mount 11 is located (i.e., the X-Y plane), and the first elastic member 131 may be parallel to a plane (i.e., a Y-Z plane) formed by the width direction of the mount 11 and the height direction of the mount 11. The direction where the second elastic member 132 is disposed may be parallel to the plane where the mount 11 is located and perpendicular to the direction where the first elastic member 131 is disposed. In some cases, since the first elastic member 131 intersects the plane where the mount 11 is located, the first elastic member 131 may at least buffer and absorb the vibration in the Z direction. Since the direction where the second elastic member 132 is disposed is parallel to the plane where the mount 11 is located, the first elastic member 131 may at least buffer the vibration in the X direction and/or the Y direction, thereby simultaneously buffering the vibration in at least two dimensions.

In some embodiments, the first elastic member 131 may intersect the plane where the mount 11 (equivalent to the X-Y plane) is located, and the first elastic member 131 may intersect the Y direction and the Z direction at the same time. The direction where the second elastic member 132 is disposed may be parallel to the length direction (equivalent to the X direction) of the mount 11.

In some cases, the first elastic member 131 may intersect the plane where the mount 11 is located, and the first elastic member 131 may intersect the Y direction and the Z direction at the same time, which aims to enable the first elastic member 131 to simultaneously buffer the vibration of the mount 11 in the Y direction (i.e., the width direction of the mount11) and the Z direction (i.e., the height direction of the mount 11). The direction where the second elastic member 132 is disposed may be parallel to the length direction of the mount 11 (equivalent to the X direction), which aim to buffer the vibration in the X direction through the second elastic member 132, so as to achieve vibration absorption of the mount 11 in three dimensions of X-Y-Z, and improve stability and safety of use of the X-ray tube (i.e., the X-ray tube 210 in FIG. 7). In addition, since the direction where the second elastic member 132 is disposed is parallel to the length direction of the mount 11, the second elastic member 132 may buffer the vibration in the X direction to a greatest extent, thereby improving the vibration absorption effect.

It should be noted that since the support unit 12 is connected to the connecting member 14 through the first elastic member 131 and the second elastic member 132, respectively, when the first elastic member 131 and the second elastic member 132 are disposed in different directions, a certain space margin may be provided for swing of the first elastic member 131 and the second elastic member 132, so that the first elastic member 131 and the second elastic member 132 may work normally. Merely by way of example, the direction where the first elastic member 131 is disposed may be located in the Z-Y plane and intersect the plane where the mount 11 is located, and the direction where the second elastic member 132 is disposed is parallel to the X direction. The second elastic member 132 may need to expand and deform in the X direction to buffer the vibration. Since the first elastic member 131 and the support unit 12 have a small swing margin in the direction where the second elastic member 132 is disposed, the second elastic member 132 may work normally. Similarly, the support unit 12 and the second elastic member 132 may also swing slightly in the direction where the first elastic member 131 is disposed, so that the first elastic member 131 may work normally.

In some embodiments, the first elastic member 131 may include a first subelastic member and a second subelastic member, both of which are located in the Y-Z plane, with the first subelastic member set in a direction parallel to the Y axis and the second subelastic member set in a direction parallel to the Z axis. The first elastic member 131 may intersect the Y direction and the Z direction at the same time, which aims to enable the first elastic member 131 to simultaneously buffer the vibration of the mount 11 in the Y direction (i.e., the width direction of the mount11) and the Z direction (i.e., the height direction of the mount 11).

Referring to FIG. 1, the embodiment provides the bearing device 100 of the X-ray tube (e.g., the X-ray tube 210 in FIG. 7), including the bottom plate, the support unit 12, and the vibration absorption unit 13. The support unit 12 may be disposed on the bottom plate and may be disposed close to the X-ray tube. The vibration absorption unit 13 may be disposed on the support unit 12 and include a first elastic member 131 and a second elastic member 132. The direction where the first elastic member 131 is disposed may be parallel to a plane where the support unit 12 is located. An inclination angle may be formed between the direction where the first elastic member 131 is disposed and the plane where the bottom plate is located. The direction where the second elastic member 132 is disposed may be perpendicular to the plane where the support unit 12 is located.

The plane where the support unit 12 is located refers to a plane determined by a length direction and a height direction of the support unit 12. The length direction of the support unit 12 may be parallel to the width direction of the bottom plate, and the height direction of the support unit 12 may be parallel to the height direction of the bottom plate, so the plane where the support unit 12 is located may be the plane determined by the height direction of the bottom plate and the width direction of the bottom plate, i.e., the Y-Z plane. The direction where the second elastic member 132 is disposed may be perpendicular to the plane where the support unit 12 is located, so the direction where the second elastic member 132 is disposed may be parallel to the length direction of the bottom plate, i.e., parallel to the X direction. The support unit 12 may be disposed close to the X-ray tube, which means that a distance between the support unit 12 and the X-ray tube is smaller than a distance threshold (e.g., 1 cm, 2 cm, 5 cm, etc.). The bottom plate in this embodiment may be equivalent to the mount in other embodiment of this specification.

It can be seen that the vibration absorption unit 13 is disposed on the bearing device 100 of the X-ray tube provided in the embodiment. The vibration absorption unit 13 may be used to relieve the vibration of the X-ray tube due to the impact force. According to the directions where the first elastic member 131 and the second elastic member 132 are disposed, in three-dimensional space, the first elastic member 131 may simultaneously achieve vibration absorption in two dimensions of the width direction of the mount 11 and the height direction of the mount 11, and the second elastic member 132 may achieve vibration absorption in another dimension (the length direction of the mount 11), so that the three degrees of freedom of movement of the X-ray tube may be constrained synchronously to realize the dynamic balance of the X-ray tube and prolong the service life of the X-ray tube.

In some embodiments, in order to effectively balance the vibration absorption effect of the first elastic member 131 on the Y direction and the Z direction, an angle between the direction where the first elastic member 131 is disposed and the plane where the mount 11 is located (equivalent to the X-Y plane) may be in a range of 30° to 60°. In some embodiments, the angle between the direction where the first elastic member 131 is disposed and the plane where the mount 11 is located may be in a range of 40° to 50°. In some embodiments, the angle between the direction where the first elastic member 131 is disposed and the plane where the mount 11 is located may be in a range of 44° to 46°. In some embodiments, the angle between the direction where the first elastic member 131 is disposed and the plane where the mount 11 is located may be 45°, so that the first elastic member 131 may buffer and absorb the vibration in the Y direction and the Z direction of a same amount.

In some embodiments, the bearing device 100 may further include a connecting member 14, and the support unit 12 is connected to the connecting member 14 through the vibration absorption unit 13. The connecting member 14 may be used to connect the bearing device 100 to other fixing devices (e.g., a support frame 15 in FIG. 1).

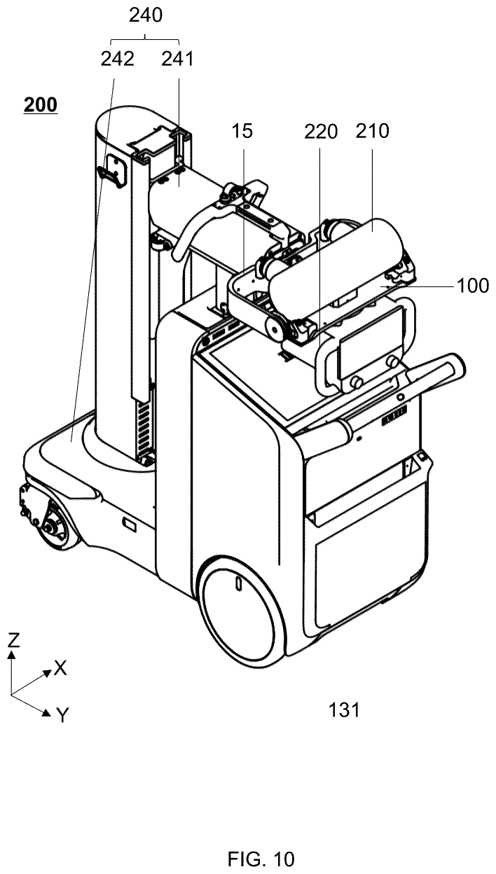

In some embodiments, the connecting member 14 may include a first connecting member 141 and a second connecting member 142. The first connecting member 141 and the second connecting member 142 may be located at two ends of a length direction of the mount 11, and the first connecting member 141 and the second connecting member 142 may be located at a same side of the mount 11. The vibration absorption unit 13 may include a first vibration absorption component 133 and a second vibration absorption component 134. The first vibration absorption component 133 may be connected to the first connecting member 141, and the second vibration absorption component 134 may be connected to the second connecting member 142. The support unit 12 may include a first support component 121 and a second support component 122. The first support component 121 may be connected to the first vibration absorption component 133, and the second support component 122 may be connected to the second vibration absorption component 134. In some embodiments, when the X-ray tube (for example, the X-ray tube 210 in FIG. 10cated at one end of the X-ray tube in the length direction, the second connecting member 142, the second vibration absorption component 134, and the second support component 122 are arranged at the other end of the X-ray tube in the length direction.

In some cases, the connecting member 14, the vibration absorption unit 13, and the support unit 12 may be disposed at the two ends of the length direction of the mount 11, which may not only further reduce vibration intensity of the X-ray tube disposed on the mount 11, but also may effectively balance the vibration at the two ends of the length direction of the mount 11, thereby further improving the stability of the mount 11.

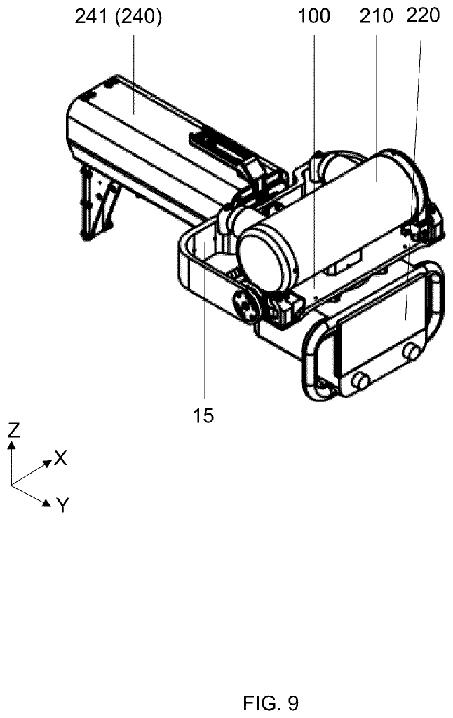

In order to avoid strong shaking of the X-ray tube due to movement of a column (e.g., a telescopic arm 241 of the mobile digital radiography device 200 in FIG. 9), the support unit 12 may be disposed on the mount 11. The support unit 12 may be vertically fixed on the mount 11 and disposed close to ends of the X-ray tube (e.g., two ends of the length direction of the X-ray tube), and may be connected to the X-ray tube through the mount 11. It may be understood that the mount 11 is disposed along the X-Y plane, and the support unit 12 is perpendicular to the mount 11, e.g., disposed along the Z-Y plane. Further, the X-ray tube may be fixedly disposed at a center of the mount 11. The support unit 12 may be spaced apart from the X-ray tube and disposed at the ends of the length direction of the mount 11. Preferably, the support unit 12 may include the first support component 121 and the second support component 122. The first support component 121 and the second support component 122 may be located on the same side of the mount 11 and may be fixedly disposed at two opposite ends of the mount 11, respectively. As shown in FIG. 1, the first support component 121 and the second support component 122 may be vertically disposed at the two opposite ends of the length direction of the mount 11. The first support component 121 and the second support component 122 may be connected to the mount 11 by riveting, welding, bolting, etc. The first support component 121 and the second support component 122 may be respectively located at the two opposite ends of the X-ray tube and may be spaced apart from the X-ray tube, which may play a blocking role when the X-ray tube is subjected to an impact force and produces a large-scale vibration, thereby reducing the vibration amplitude of the X-ray tube and improving the stability of the X-ray tube.

In order to further improve the vibration absorption effect of the support unit 12 on the X-ray tube, the vibration absorption unit 13 may be disposed on the support unit 12. The vibration absorption unit 13 may include a first elastic member 131 and a second elastic member 132. Two opposite ends of the first elastic member 131 may be installed in the support unit 12. Two opposite ends of the second elastic member 132 may be installed in the support unit 12. One end of the second elastic member 132 may extend towards the X-ray tube and may be spaced apart from the X-ray tube. The direction where the first elastic member 131 is disposed may be parallel to the plane where the support unit 12 is located and an inclination angle may be formed between the direction where the first elastic member 131 is disposed and the plane where the mount 11 is located, i.e., the first elastic member 131 may be disposed in the Z-Y plane. The direction where the second elastic member 132 is disposed may be perpendicular to the plane where the support unit 12 is located, i.e., the second elastic member 132 may be disposed in a Z-X plane. According to this, in actual operation, the vibration transmitted when the moving device of the mobile digital radiography device crosses a threshold may be decomposed into three directions of X-Y-Z, the first elastic member 131 disposed obliquely may absorb the vibration in the Y direction and the Z direction, the second elastic member 132 disposed horizontally may absorb the vibration in the X direction, which may effectively control a maximum impact acceleration within an allowable range, realize the synchronous constraint of the three degrees of freedom of movement of the X-ray tube, ensure the dynamic balance of the X-ray tube, prolong the service life of the X-ray tube, and improve the safety of use of the mobile digital radiography device.

In some embodiments, each of the first vibration absorption component 133 and the second vibration absorption component 134 may include at least one first elastic member 131 and at least one second elastic member 132, so as to simultaneously buffer the vibration at the two ends of the length direction of the mount 11 and make the two ends of the length direction of the mount 11 balanced. In some embodiments, at least one of the first vibration absorption component 133 and the second vibration absorption component 134 may include two or more first elastic members 131. In some embodiments, at least one of the first vibration absorption component 133 and the second vibration absorption component 134 may include two or more second elastic members 131.

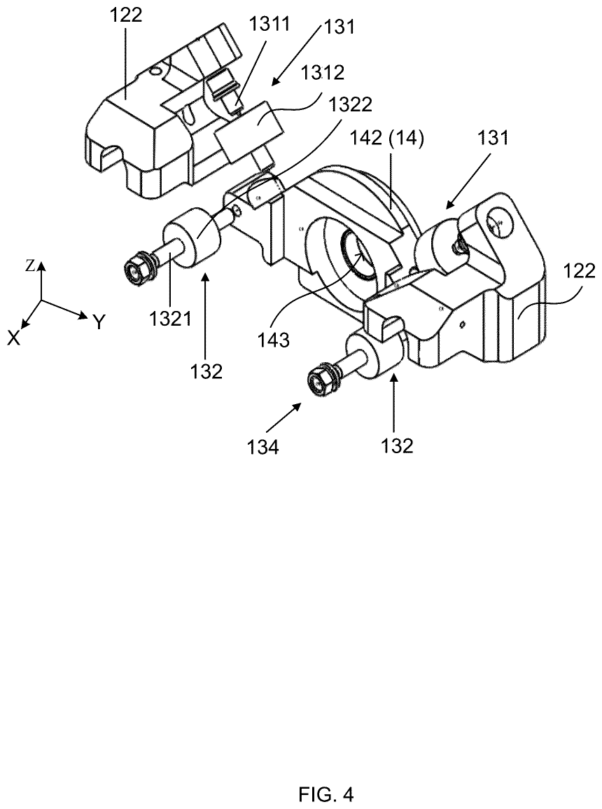

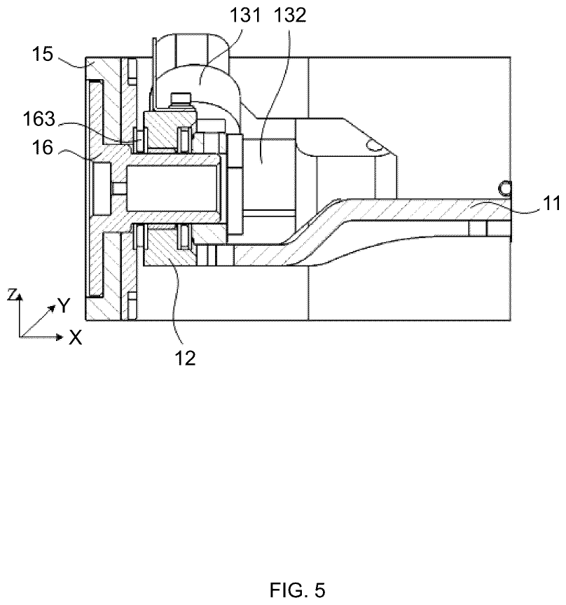

In some embodiments, a count of first elastic members 131 and a count of second elastic members 132 in the first vibration absorption component 133 may be the same. For example, the first vibration absorption component 133 may include a first elastic member 131 and a second elastic member 132. As another example, as shown in FIG. 4 and FIG. 5, the first vibration absorption component 133 may include two first elastic members 131 and two second elastic members 132. Similarly, in some embodiments, as shown in FIG. 3, a count of first elastic members 131 and a count of second elastic members 132 in the second vibration absorption component 134 may be the same.

In some embodiments, the count of the first elastic members 131 and the count of the second elastic members 132 in the first vibration absorption component 133 may be different. For example, the first vibration absorption component 133 may include three first elastic members 131 and two second elastic members 132. Similarly, in some embodiments, the count of first elastic members 131 and the count of second elastic members 132 in the second vibration absorption component 134 may be different.

Further, in some embodiments, the first elastic member 131 may be disposed at an angle relative to the mount 11, which aims to provide a certain vibration absorption effect on the X-ray tube in the X direction and the Z direction. Therefore, when the two or more first elastic members 131 are disposed on at least one of the first support component 121 and the second support component 122, an angle may be formed between directions where at least two of the two or more first elastic members are disposed. It may be understood that at least two of the two or more first elastic members 131 are parallel to each other, and extension lines of the directions where at least two of the two or more first elastic members 131 are disposed intersect, so as to further improve the vibration absorption effect in the X direction and the Z direction. For example, three first elastic members 131 may be disposed on each of the first support component 121 and the second support component 201. Taking the three first elastic members 131 on the first support component 121 as an example, the three first elastic members 131 may be spaced apart on the first support component 121. Two of the first elastic members 131 may be parallel to each other, and an angle may be formed between directions where the two first elastic members 131 are disposed and a direction where a third first elastic member 131 is disposed; or extension lines of the three first elastic members 131 may intersect. The situation that the two or more first elastic members 131 are disposed on at least one of the first support component 121 and the second support component 122 is described in detail below with reference to the accompanying drawings.

In some embodiments, when at least one of the first vibration absorption component 133 and the second vibration absorption component 134 includes the two or more first elastic members 131, the two or more first elastic members 131 may be located in a same plane. In some embodiments, when there are two or more first elastic members 131, at least one first elastic member 131 and other first elastic members 131 may be located in different planes.

In some embodiments, at least one of the first vibration absorption component 133 and the second vibration absorption component 134 may include the two or more first elastic members 131. An angle greater than 0 degrees and smaller than 180 degrees may be formed between directions where at least two of the two or more first elastic members 131 of the first vibration absorption component 133 are disposed. In some embodiments, an angle greater than 0 degrees and smaller than 180 degrees is formed between directions where at least two of the two or more first elastic members 131 of the second vibration absorption component 134 are disposed. Merely by way of example, taking the first vibration absorption component 133 as an example, the first vibration absorption component 133 may include three first elastic members 131. Two of the three first elastic members 131 may be located in a same plane (e.g., a plane where a Z-Y direction is located) and parallel to each other (e.g., parallel to the Z direction). The remaining first elastic member 131 of the three first elastic members 131 may be located in the Z-X plane and an angle of 45 degrees may be formed between a direction where the third first elastic member 131 is disposed and the Z direction. As another example, the directions where the three first elastic members 131 are disposed may intersect.

In order to ensure force balance, preferably, a count of first elastic members 131 disposed on the first support component 121 may be equal to a count of first elastic members 131 disposed on the second support component 122. A count of second elastic members 132 disposed on the first support component 121 may be equal to a count of second elastic members 132 disposed on the second support component 122. The first elastic members 131 and the second elastic members 132 may be disposed in symmetrical directions and positions. For example, one first elastic member 131 and one second elastic member 132 may be disposed in the first support component 121, and similarly, one first elastic member 131 and one second elastic member 132 may also be disposed on the second support component 122 at opposite positions.

In some embodiments, the second elastic member 132 may be disposed along a direction perpendicular to the plane where the support unit 12 is located, which aims to achieve vibration absorption of the X-ray tube in the X direction. Preferably, when two or more second elastic members 132 are disposed on at least one of the first support component 121 and the second support component 122, in order to ensure uniform force, a count of second elastic members 132 disposed on the first support component 121 may be the same as a count of second elastic members 132 disposed on the second support component 12, and positions where the second elastic members 132 are disposed on the first support component 121 may correspond to positions where the second elastic members 132 are disposed on the second support component 121. Moreover, each of the second elastic members 132 may be spaced apart. Preferably, a plurality of first elastic members 131 and a plurality of second elastic members 132 may be disposed on the first support component 121 and the second support component 122, so that when a part of the first elastic members 131 and/or the second elastic members 132 are damaged, the remaining first elastic members 131 and/or the remaining second elastic members 132 may still work normally, and the overall structure may not lose the function. The positions where the first elastic member 131 and the second elastic member 132 are specifically disposed and the specific counts of the first elastic member 131 and the second elastic member 132 may not be limited in the embodiment.

In the example shown in FIGS. 1-2 provided in the embodiment, two first elastic members 131 and two second elastic members 132 may be disposed on each of the first support component 121 and the second support component 122. The two first elastic members 131 and the two second elastic members 132 may be symmetrically disposed on the corresponding first support component 121 and the second support component 122, respectively, so as to enhance dynamic stability of the X-ray tube. In order to balance and control the vibration absorption effect in the X direction and the Z direction, the inclination angle between the direction where the first elastic member 131 is disposed and the X-Y plane where the mount 11 is located may be in a range of 44°-46°, i.e., an angle between the direction where the first elastic member 131 is disposed and the X-Y plane may be in a range of 44° to 46°, preferably, 45°, so that the vibration in the Y direction and the Z direction of a same amount may be buffered. Further, the directions where the two first elastic members 131 are disposed on at least one of the first support component 121 and the second support component 122 may be perpendicular to each other, so as to further balance the impact force on the X-ray tube in the Y direction and the Z direction and ensure the stability in use of the X-ray tube.

As shown in FIG. 2, the first support component 121 and the second support component 122 have a support body. The supporting body may not only play a role of connecting the mount 11, but also be used to bear and support the first elastic member 131 and the second elastic member 132 and cooperate with connection of two opposite ends of the first elastic member 131 and the second elastic member 132, which can ensure the stability of the first elastic member 131 and the second elastic member 132 and improve device performance. The first elastic member 131 and the second elastic member 132 may be detachably connected to the support body, e.g., through threaded connection or snapping, and the support body may be spaced apart from the X-ray tube, so as to facilitate disassembly and replacement of a damaged first elastic member 131 and second elastic member 132. Optionally, a material used to make the support body may include, but is not limited to, metal, polymer, or carbon fiber. In some embodiments, the support body may be equivalent to the connecting member 14 in other embodiments of the present disclosure. The first support component, the second support component, and the connecting member 14 are described in detail below with reference to FIGS. 1-2.

In some embodiments, a count of the at least one first elastic member 131 of the first vibration absorption component 133 may be the same as a count of the at least one first elastic member 131 of the second vibration absorption component 134. The at least one first elastic member of the first vibration absorption component and the at least one first elastic member of the second vibration absorption component may be disposed symmetrically at the two ends of the length direction of the mount 11. Similarly, in some embodiments, a count of the at least one second elastic member 132 of the first vibration absorption component 133 may be the same as a count of the at least one second elastic member 132 of the second vibration absorption component 134. The at least one second elastic member of the first vibration absorption component and the at least one second elastic member of the second vibration absorption component may be disposed symmetrically at the two ends of the length direction of the mount 11. The at least one first elastic member of the first vibration absorption component and the at least one first elastic member of the second vibration absorption component being disposed symmetrically means that any part of the two elastic members is symmetrical with respect to a specific plane. For example, a line connecting midpoints of the length direction of the mount may extend along the width direction of the mount, and a plane perpendicular to the mount and containing the line connecting midpoints may be called a midline plane. The at least one first elastic member of the first vibration absorption component and the at least one first elastic member of the second vibration absorption component being disposed symmetrically means that one end of the first elastic member 131 of the first vibration absorption component 133 and one end of the first elastic member 131 of the second vibration absorption component 134 may be disposed symmetrically with respect to the midline plane, and the other end of the first elastic member 131 of the first vibration absorption component 133 and the other end of the first elastic member 131 of the second vibration absorption component 134 may be also disposed symmetrically with respect to the midline plane.

In some cases, the count of the at least one first elastic member 131 of the first vibration absorption component 133 may be set the same as the count of the at least one first elastic member 131 of the second vibration absorption component 134, and the at least one first elastic member of the first vibration absorption component and the at least one first elastic member of the second vibration absorption component may be disposed symmetrically at the two ends of the length direction of the mount 11, which may make the at least one first elastic member 131 at the two ends of the length direction of the mount 11 have a similar vibration absorption effect and further ensure that the vibration at the two ends of the length direction of the mount 11 tends to be balanced, thereby improving the stability of the mount 11 and reducing the impact force on the X-ray tube (e.g., the X-ray tube 210 in FIG. 7). Similarly, the count of the at least one second elastic member 132 of the first vibration absorption component 133 may be set the same as the count of at least one second elastic member 132 of the second vibration absorption component 134, and the at least one second elastic member of the first vibration absorption component and the at least one second elastic member of the second vibration absorption component may be disposed symmetrically at the two ends of the length direction of the mount 11, which may make the at least one second elastic member 132 at the two ends of the length direction of the mount 11 have a similar vibration absorption effect and further ensure that the vibration at the two ends of the length direction of the mount 11 tends to be balanced, thereby improving the stability of the mount 11 and reducing the impact force on the X-ray tube.

In some embodiments, at least one of the first vibration absorption component 133 and the second vibration absorption component 134 may include two second elastic members 132 and two first elastic members 131. The two first elastic members 131 may be disposed perpendicular to each other. Merely by way of example, taking the second vibration absorption component 134 in FIG. 3 as an example, the second vibration absorption component 134 includes the two first elastic members 131 and the two second elastic members 132. The two first elastic members 131 may be located in the Y-Z plane, an angle of 45° may be formed between each of the two first elastic members 131 and the X-Y plane, and the directions where the two first elastic members 132 are disposed may intersect, so that the two first elastic members 131 may be disposed perpendicular to each other. Each of the two first elastic members 131 in the embodiment may be capable of buffering the vibration in the Z direction and the Y direction, so as to further balance the impact force on the X-ray tube in the Y direction and Z-direction and further ensure the stability in use of the X-ray tube (e.g., the X-ray tube 210 in FIG. 7).

In some embodiments, the two first elastic members 131 of the first vibration absorption component 133 may be disposed symmetrically at two ends of a length direction of the first connecting member 141, and the two second elastic members 132 of the first vibration absorption component 133 may be disposed symmetrically at the two ends of the length direction of the first connecting member 141; and/or the two first elastic members 131 of the second vibration absorption component 134 may be disposed symmetrically at two ends of a length direction of the second connecting member 142, and the two second elastic members 132 of the second vibration absorption component 134 may be disposed symmetrically at the two ends of the length direction of the connecting member 142. Merely by way of example, the second connecting member 142 and the second vibration absorption component 134 in FIG. 3 are taken as examples for illustration. The second connecting member 142 may be a plate-like structure, and a length direction of the second connecting member 142 may be parallel to the width direction of the mount 11, and a width direction of the second connecting member 142 may be parallel to the length direction of the mount 11. The two first elastic members 131 of the second vibration absorption component 134 may be disposed at the two ends of the length direction of the second connecting member 142. The two first elastic members 131 may be located in a plane defined in the Y direction and Z direction, and an angle of 45° may be formed between each of the two first elastic members 131 and the X-Y plane. Extension lines of the directions where the two first elastic members 131 are disposed may intersect. The two second elastic members 132 of the second vibration absorption component 134 may be disposed symmetrically at the two ends of the length direction of the second connecting member 142, and the directions where the two second elastic members 132 are disposed may be parallel to the length direction of the mount 11.

In some cases, the two first elastic members 131 of the first vibration absorption component 133 may be disposed symmetrically at the two ends of the length direction of the first connecting member 141, which may balance the vibration (e.g., vibration in the Y direction and Z direction) at the two ends of the length direction of the first connecting member 141 to further improve stability. Similarly, the two second elastic members 132 of the first vibration absorption component 133 may be disposed symmetrically at the two ends of the length direction of the second connecting member 142, which may balance the vibration (e.g., vibration in the Y direction) at the two ends of the length direction of the second connecting member 142 to balance to further improve stability.

In some embodiments, the first elastic member 131 and the second elastic member 132 may be detachably connected to the connecting member 14 (e.g., through threaded connection, snapping, magnetic connection), so that the first elastic member 131 and/or the second elastic member 132 may be replaced when damaged. More descriptions regarding the connection manner of the first elastic member 131, the second elastic member 132, and the connecting member 14 may be found in FIG. 3-FIG. 5 and the embodiments thereof, which will not be repeated herein.

Further, in some embodiments, since a weight of the bearing device of the X-ray tube also has a certain role in promoting the transmission of vibration, in order to reduce the weight of the bearing device of the X-ray tube, the bearing device of the X-ray tube in the embodiment may be preferably made of a lightweight material such as carbon fiber.

In some embodiments, the connecting member may be used to support the first elastic member 131 and the second elastic member 132 to ensure the stability of the first elastic member 131 and the second elastic member 132. In some embodiments, the material used to make the connecting member 14 may include, but is not limited to metal, polymer, or carbon fiber.

In some embodiments, the first elastic member 131 may include a first rigid column 1311 and a first elastic element 1312 sleeved on the first rigid column 1311. The second elastic member 132 may include a second rigid column 1321 and a second elastic ring 1322 sleeved on the second rigid column 1321. The first elastic element 1312 may be connected to the support unit 12 through the first rigid column 1311, and two ends of the first elastic element 1312 may be in contact with the support unit 12 and the connecting member 14, respectively. The second elastic ring 1322 may be connected to the support unit 12 through the second rigid column 1321, and two ends of the second elastic ring 1322 may be in contact with the support unit 12 and the connecting member 14, respectively. In the embodiment, since the two ends of the first elastic element 1312 may be in contact with the support unit 12 and the connecting member 14, respectively, vibration transmitted from the connecting member 14 to the support unit 12 may be reduced. In addition, since the first elastic element 1312 is connected to the support unit 12 through the first rigid column 1311, the strength and stability of the connection between the first elastic element 1312 and the connecting member 14 may be effectively improved. Similarly, since the two ends of the second elastic ring 1322 may be in contact with the support unit 12 and the connecting member 14, respectively, vibration transmitted from the connecting member 14 to the support unit 12 may also be reduced. Since the second elastic ring 1322 is connected to the support unit 12 through the second rigid column, the strength and stability of connection between the second elastic ring 1322 and the connecting member 14 may be effectively improved.

In some embodiments, the first rigid column 1311 and the second rigid column 1321 may be fixedly connected or detachably connected to the support unit 12. Exemplary fixed connection manners may include riveting, bonding, welding, etc. Exemplary detachable connection manners may include threaded connection, snap connection, etc.

In some embodiments, the first rigid column 1311 and the second rigid column 1321 may each include a metal column. In some embodiments, the first elastic element 1312 and the second elastic element may include a ring structure, a sleeve structure, a barrel structure, etc. In some embodiments, the materials for making the first elastic element 1312 and the second elastic element may include rubber, plastic, etc. In some embodiments, the first elastic element 1312 and the second elastic ring may each include a rubber ring. In some embodiments, the first elastic element 1312 and the second elastic element may include a rubber sleeve, a plastic ring, a plastic sleeve, etc.

In some cases, the rubber ring may be relatively light in weight and have good damping and pressure resistance, so the rubber ring may be capable of effectively absorbing vibration energy. In addition, after the first elastic member 131 and the second elastic member 132 are added to the two ends of the length direction of the X-ray tube (e.g., the X-ray tube 210 in FIG. 7), the complexity of vibration absorption measures of other associated components (e.g., the support unit 12, the connecting member 14) may be reduced, which can effectively optimize the structure of the whole machine system. Merely by way of example, at least one end of the metal column may be provided with threads, for example, the metal column may be a Q235 metal stud, so as to be threadedly connected with the support unit 12. Since the threaded connection structure may be relatively simple, it is easy to installed without adding other auxiliary devices. In addition, a vibration unit or the support unit 12 may be directly replaced when failing, which is more convenient and quicker.

In some embodiments, the support unit 12 may be formed by connecting a plurality of independent parts. Merely by way of example, the support unit 12 may include a first support body connected to the first elastic member 131 and the mount 11, and a second support body connected to the second elastic member 132 and the mount 11, and the first support body may be connected to the second support body. In some embodiments, the first support body and the second support body may be elastically connected, e.g., the first support body and the second support body may be connected through an elastic structure, so as to reduce vibration of the support unit 12, thereby reducing the vibration transmitted to the mount 11. In some embodiments, the support unit 12 may be integrally formed to increase the overall stability of the support unit 12.

In some embodiments, loss during the vibration transmission may be increased by reducing a weight of the support unit 12. In some embodiments, a material used to make the support unit 12 may include aluminum and carbon fiber.

In some embodiments, the mount 11 may be a rectangular or rounded rectangular plate. In some embodiments, a material used to make the mount 11 may include, but is not limited to, metal, polymer, or carbon fiber.

In some embodiments, the mount 11 may be detachably connected to the support unit 12, so as to replace the support unit 12. Merely by way of example, the mount 11 may be provided with a plurality of screw holes, and the support unit 12 may be detachably connected to the mount 11 through bolts. More descriptions regarding the detachable connection manner may be found in other embodiments of the present disclosure. In some embodiments, the mount 11 may be fixedly connected to the support unit 12.

In some embodiments, as shown in FIGS. 1-2, the bearing device 100 may include a support frame 15. The connecting member 14 may be disposed on the two ends of the length direction of the mount 11, and two opposite ends of the support frame 15 may be connected to the connecting member 14. In some embodiments, the support frame 15 may be fixedly connected or detachably connected to the connecting member 14. More descriptions regarding the connection manner of fixed connection or detachable connection may be found in other embodiments of the present disclosure.

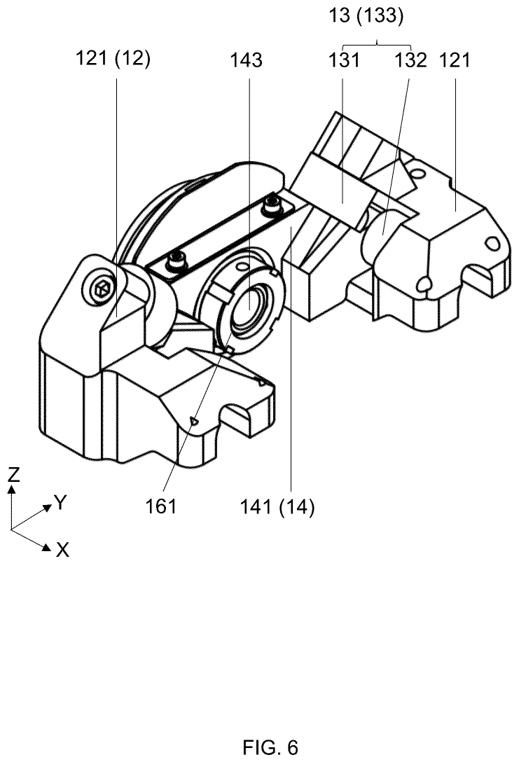

In some embodiments, as shown in FIGS. 1-6, the bearing device 100 may further include the support frame 15 and a support shaft 16. The connecting member 14 may be disposed on the two ends of the length direction of the mount 11, and the two opposite ends of the support frame 15 may be rotatably connected to the connecting member 14 through the support shaft 16. Merely by way of example, the support shaft 16 may include a first support shaft 161 and a second support shaft 162, and each of the first connecting member 141 and the second connecting member 142 may be provided with a mounting hole 143, and the mounting hole 143 may be used for the support shaft 16 to pass through. The two mounting holes 143 may be coaxially disposed and parallel to the length direction of the mount 11. Outer surfaces of the first support shaft 161 and the second support shaft 162 may be provided with needle roller components 163. The first support shaft 161 may pass through the first connecting member 141 and one end of the support frame 15, so that the first connecting member 141 and the first support shaft 161 may be relatively fixed through cooperation of the R-shaped lock nut (not shown in the figure) and the needle roller component 163, and the first support shaft 161 may rotate relative to the one end of the support frame 15, thereby driving the mount 11 to rotate around the first support shaft 161. Similarly, the second support shaft 162 may pass through the second connecting member 142 and the other end of the support frame 15, so that the second connecting member 142 and the second support shaft 162 may be opposed to each other through the cooperation of the R-shaped lock nut and the needle roller component 163, and the second support shaft 162 may rotate relative to the other end of the support frame 15, thereby driving the mount 11 to rotate around the second support shaft 162. Since the mounting holes 143 on the first connecting member 141 and the second connecting member 142 are disposed coaxially, when rotating simultaneously, the first support shaft 161 and the second support shaft 162 may be capable of driving the mount 11 to rotate. In some practical application scenarios, when the X-ray tube (e.g., the X-ray tube 210 in FIG. 7) is installed on the mount 11, a beam direction of the X-ray tube may be parallel to a thickness direction (perpendicular to the X direction and the Y direction) of the mount 11, and the mount 11 may be driven to rotate by rotating the first support shaft 161 and the second support shaft 162, thereby adjusting an inclination angle between a line connecting two ends of the width direction of the mount 11 and a horizontal plane and then adjusting an angle between the beam emitted by the X-ray tube and the Y direction.

In some embodiments, the bearing device 100 may further include a connecting shaft 17. The connecting shaft 17 may be disposed on the support frame 15, which may be used to connect the support frame 15 to an external component (e.g., a C-arm). In some embodiments, an axial direction of the connecting shaft 17 may be parallel to the width direction of the mount 11, one end of the connecting shaft 17 may be rotatably connected to the support frame 15, and the other end may be fixedly connected to a mechanical arm, and the axial direction of the connecting shaft 17 may be perpendicular to an axial direction of the support shaft 16 (e.g., the first support shaft 161 and the second support shaft 162). After the mount 11 is installed on the support frame 15, an inclination angle between a line connecting two ends of the length direction of the mount 11 and the horizontal plane may be adjusted by rotating the connecting shaft 17. In some practical application scenarios, when the X-ray tube is installed on the mount 11, the mount 11 may be driven to rotate by rotating the connecting shaft 17, thereby adjusting an angle between the beam emitted by the X-ray tube and the X direction. In some embodiments, the axial direction of the connecting shaft 17 may be located at a midpoint in the length direction of the mount 11.

In some embodiments, the bearing device of the X-ray tube may further include the support frame 15, the support shaft 16, and the connecting shaft 17. The support frame 15 may be used to support the X-ray tube. A main body of the X-ray tube may be located on the mount 11, and remaining protruding parts may abut on the support frame 15, the support frame 15 may be used to further fix the X-ray tube. Two opposite ends of the support frame 15 may be located at two opposite ends of the mount 11, respectively, and the support shaft 16 may pass through an end of the support frame 15 and may be disposed in the support unit 12 to fix the support frame 15 to the support unit 12. As shown in FIGS. 1-3, FIG. 3 is a cross-sectional view of the first support component 121 along A-A′ in FIG. 1. The two ends of the support frame 15 may be respectively fixed in the first support component 121 and the second support component 122 through the corresponding support shaft 16. Moreover, each of central positions of the first support component 121 and the second support component 122 may be provided with a second hole, which may be used for the support shaft 16 to be disposed in the support unit 12. In some embodiments, the second hole may be equivalent to the mounting hole 143 in other embodiments of the present disclosure. Further, each outer surface of the support shaft 16 may be provided with the needle roller component 163 and fixed in the corresponding first support component 121 and the second support component 122 by an R-shaped lock nut for driving the support unit 12, the mount 11, and the X-ray tube to rotate synchronously when the support shaft 16 rotates in the axial direction, so as to adjust a position of the X-ray tube. The connecting shaft 17 may be disposed on the support frame 15, which may be used to connect the support frame 15 to the external component.

According to the same inventive concept, the embodiment also provides a mobile digital radiography imaging device, including the bearing device of the X-ray tube and the X-ray tube, and the X-ray tube may be installed on the bearing device of the X-ray tube. In some embodiments, the mobile digital radiography imaging device may be equivalent to the mobile digital radiography device in other embodiments of the present disclosure.

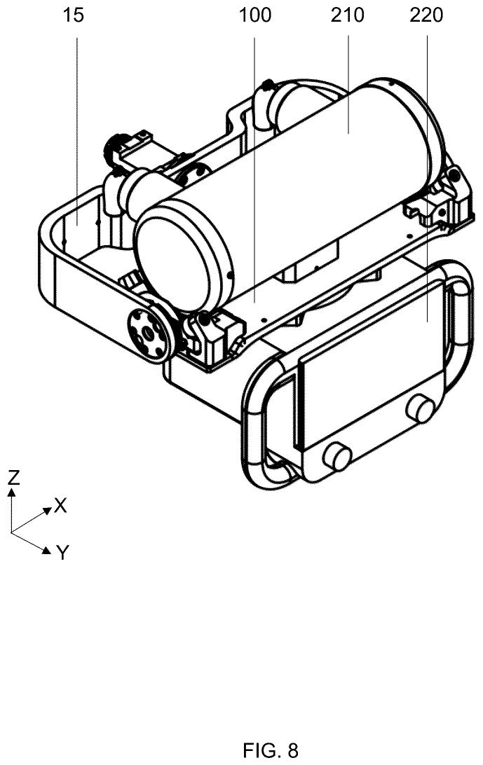

The present disclosure also provides a mobile digital radiography device. As shown in FIG. 1 and FIGS. 7-10, the mobile digital radiography device 200 may include an X-ray tube 210, a beam limiter 220, and the bearing device 100 of the X-ray tube 210 in the one or more embodiments, and the X-ray tube 210 may be installed on the bearing device 100 of the X-ray tube 210. In some embodiments, the X-ray tube 210 may be disposed on one side of the mount 11, and a hole 111 may be disposed at a position corresponding to the X-ray tube 210 of the mount 11 for a beam of the X-ray tube 210 to pass through. The beam limiter 220 may be disposed on the other side of the mount 11 opposite to the X-ray tube 210 and may be configured to receive the beam passing through the hole 111. The beam limiter 220 may form a radiation field of a specific shape, which may limit an irradiation range of the beam.

In some embodiments, the X-ray tube 210 may be fixedly disposed on the mount 11, for example, the X-ray tube 210 may be welded or bonded to the mount 11. In some embodiments, the X-ray tube 210 may be detachably disposed on the mount 11, for example, the X-ray tube 210 may be disposed on the mount 11 through threaded connection, bonding, snapping, magnetic connection, etc. Merely by way of example, as shown in FIG. 1 and FIG. 7, a plurality of threaded holes may be disposed around the hole 111 of the mount 11 so as to connect the X-ray tube 210 to the mount 11 by bolts.

In some embodiments, the X-ray tube may be disposed along the length direction of the mount 11, i.e., along the X direction. A first hole may be disposed at a central position of the mount 11 and may be configured to facilitate penetrating of the beam of the X-ray tube. The plurality of threaded holes may be disposed around the first hole so as to fixedly connect the X-ray tube to the mount 11 by the bolts. In some embodiments, the first hole may be equivalent to the hole 111 in other embodiments of the present disclosure.

In some embodiments, the mobile digital radiography device 200 may further include an attitude sensor 230 configured to detect an attitude of the X-ray tube 210. In some embodiments, the attitude sensor 230 may include a three-axis gyroscope, a three-axis accelerometer, etc. Merely by way of example, taking the three-axis gyroscope as an example, the three-axis gyroscope may detect an attitude angle of the X-ray tube 210, and the attitude angle may include a pitch angle and a roll angle. The pitch angle refers to an angle between a direction of the beam emitted by the X-ray tube 210 and the Y direction. The roll angle refers to an angle between the direction of the beam emitted by the X-ray tube 210 and the X direction. In some cases, the beam direction of the X-ray tube 210 may be detected by disposing the attitude sensor 230, thereby helping an operator to control the beam emitted by the X-ray tube 210 more precisely.

In some embodiments, the mobile digital radiography device 200 may further include a moving device 240, and the bearing device may be connected to the moving device 240 through the support frame (e.g., the support frame 15 in FIG. 1), so as to drive the X-ray tube 210 through the moving device 240. In some embodiments, the moving device 240 may include the telescopic arm 241, and the telescopic arm 241 may be connected to the connecting shaft (e.g., the connecting shaft 17 in FIG. 1) and telescopically move in the width direction of the mount (e.g., the mount 11 in the FIG. 1), thereby driving the X-ray tube 210 to move along the width direction of the mount. In some embodiments, the moving device 240 may include a moving base 242, the moving base 242 may be connected to the telescopic arm 241, and the moving base 242 may move horizontally on the ground, thereby driving the X-ray tube 210 to move horizontally.

To sum up, the embodiment provides the bearing device 100 of the X-ray tube and the mobile digital radiography imaging device. The vibration absorption unit 13 may be disposed on the bearing device 100 of the X-ray tube. The vibration absorption unit 13 may be used to relieve the vibration of the X-ray tube due to the impact force. Further, the first elastic member 131 and the second elastic member 132 may be disposed on the vibration absorption unit 13; the direction where the first elastic member 131 is disposed may be parallel to the plane where the support unit 12 is located, and the inclination angle may be formed between the direction where the first elastic member 131 is disposed and the plane where the mount 11 is located; and the direction where the second elastic member 132 is disposed may be perpendicular to the plane where the support unit 12 is located. Therefore, in a three-dimensional space, the first elastic member 131 may simultaneously realize the vibration absorption of two dimensions, and the second elastic member 132 may realize the vibration absorption in another dimension, so that the degrees of freedom of movement of the X-ray tube may be synchronously constrained, which can realize the dynamic balance of the X-ray tube, prolong the service life of the X-ray tube, and improve the safety of use of the mobile digital radiography imaging device.

The beneficial effects of the bearing device and the mobile digital radiography device in the present disclosure may include but are not limited to the followings: (1) since the first elastic member and the second elastic member may be disposed in different directions, the first elastic member and the second elastic member may absorb and buffer the vibration in different directions (e.g., the directions where the first elastic member and the second elastic member are disposed), and moreover, since the vibration may be decomposed into components in three directions, by setting the first elastic member and the second elastic member to absorb and buffer the vibration in different directions, the vibration absorption on the plurality of degrees of freedom of the X-ray tube can be simultaneously performed, which realizes the dynamic balance of the X-ray tube, prolongs the service life of the X-ray tube, and improves the safety of use of the mobile digital radiography device; (2) the first elastic member may intersect with the plane where the mountis located and the first elastic part may intersect with the Y direction and the Z direction simultaneously, which aims to enable the first elastic part to simultaneously buffer the vibration of the mount in the Y direction (i.e., the width direction of the mount) and in the Z direction (i.e., the height direction of the mount), and the direction where the second elastic member is disposed may be parallel to the length direction (equivalent to the X direction) of the mount, which aims to buffer the vibration in the X direction through the second elastic member, so as to realize the vibration absorption of the mount in the three dimensions of X-Y-Z and improve the stability and safety of use of the X-ray tube; (3) the connecting member, the vibration absorption unit, and the support unit may be disposed at the two ends of the length direction of the mount, which can not only further reduce the vibration intensity of the X-ray tube installed on the mount, but also effectively balance the vibration at the two ends of the length direction of the mount, thereby further improving the stability of the mount; (4) the count of the at least one first elastic member of the first vibration absorption component may be set the same as the count of the at least one first elastic member of the second vibration absorption component, and the at least one second elastic member of the first vibration absorption component and the at least one second elastic member of the second vibration absorption component may be disposed symmetrically at the two ends of the length direction of the mount, which may make the at least one first elastic member at the two ends of the length direction of the X-ray tube have a similar vibration absorption effect, thereby further improving the stability of the mount and reducing the impact force on the X-ray tube; (5) when the X-ray tube is installed on the mount, the beam direction of the X-ray tube may be parallel to the thickness direction (perpendicular to the X direction and Y direction) of the mount, and the mount may be driven to rotate by rotating the first support shaft and the second support shaft, so as to adjust the inclination angle between the line connecting the two ends of the width direction of the mount and the horizontal plane and then adjust the angle (i.e., the pitch angle) between the beam emitted by the X-ray tube and the Y direction; and (6) the connecting shaft may be disposed, which may drive the mount to rotate by rotating the connecting shaft when the X-ray tube is installed on the mount, thereby adjusting the angle (i.e., roll angle) between the beam emitted by the X-ray tube and the X direction.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, some features, structures, or features in the present disclosure of one or more embodiments may be appropriately combined.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A bearing device (100), comprising a mount (11), a support unit (12), and a vibration absorption unit (13), wherein
the mount (11) is connected to the support unit (12);
the support unit (12) is connected to the vibration absorption unit (13); and
the vibration absorption unit (13) includes at least one elastic member (130).

2. The bearing device (100) of claim 1, wherein the vibration absorption unit (13) includes a first elastic member (131) and a second elastic member (132), the first elastic member (131) and the second elastic member (132) being disposed in different directions.

3. The bearing device (100) of claim 2, wherein a direction where the first elastic member (131) is disposed is perpendicular to a direction where the second elastic member (132) is disposed.

4. The bearing device (100) of claim 3, wherein
the direction where the first elastic member (131) is disposed intersects a plane where the mount (11) is located; and
the direction where the second elastic member (132) is disposed is parallel to a length direction of the mount (11).

5. The bearing device (100) of claim 4, wherein an angle between the direction where the first elastic member (131) is located and the plane where the mount (11) is located is in a range of 30°-60°.

6. The bearing device (100) of claim 1, further comprising a connecting member (14), wherein the support unit (12) is connected to the connecting member (14) through the vibration absorption unit (13).

7. The bearing device (100) of claim 6, wherein
the connecting member (14) includes a first connecting member (141) and a second connecting member (142), the first connecting member (141) and the second connecting member (142) being located at two ends of a length direction of the mount (11), and the first connecting member (141) and the second connecting member (142) being located at a same side of the mount (11);
the vibration absorption unit (13) includes a first vibration absorption component (133) and a second vibration absorption component (134), the first vibration absorption component (133) being connected to the first connecting member (141), and the second vibration absorption component (134) being connected to the second connecting member (142); and
the support unit (12) includes a first support component (121) and a second support component (122), the first support component (121) being connected to the first vibration absorption component (133), and the second support component (122) being connected to the second vibration absorption component (134).

8. The bearing device (100) of claim 7, wherein each of the first vibration absorption component (133) and the second vibration absorption component (134) includes at least one first elastic member (131) and at least one second elastic member (132).

9. The bearing device (100) of claim 8, wherein
at least one of the first vibration absorption component (133) and the second vibration absorption component (134) includes two or more first elastic members (131); and
an angle greater than 0 degrees and smaller than 180 degrees is formed between directions where at least two of the two or more first elastic members (131) of the first vibration absorption component (133) are disposed; and/or
an angle greater than 0 degrees and smaller than 180 degrees is formed between directions where at least two of the two or more first elastic members (131) of the second vibration absorption component (134) are disposed.

10. The bearing device (100) of claim 8, wherein
a count of the at least one first elastic member (131) of the first vibration absorption component (133) is the same as a count of the at least one first elastic member (131) of the second vibration absorption component (134), the at least one first elastic member (131) of the first vibration absorption component (133) and the at least one first elastic member (131) of the second vibration absorption component (134) being disposed symmetrically at the two ends of the length direction of the mount (11); and
a count of the at least one second elastic member (132) of the first vibration absorption component (133) is the same as a count of the at least one second elastic member (132) of the second vibration absorption component (134), the at least one second elastic member (132) of the first vibration absorption component (133) and the at least one second elastic member (132) of the second vibration absorption component (134) being disposed symmetrically at the two ends of the length direction of the mount (11).

11. The bearing device (100) of claim 8, wherein at least one of the first vibration absorption component (133) and the second vibration absorption component (134) includes two first elastic members (131) and two second elastic members (132), the two first elastic members (131) being disposed perpendicular to each other.

12. The bearing device (100) of claim 11, wherein
the two first elastic members (131) of the first vibration absorption component (133) are disposed symmetrically at two ends of a length direction of the first connecting member (141), and the two second elastic members (132) of the first vibration absorption component (133) are disposed symmetrically at the two ends of the length direction of the first connecting member (141); and/or
the two first elastic members (131) of the second vibration absorption component (134) are disposed symmetrically at two ends of a length direction of the second connecting member (142), and the two second elastic members (132) of the second vibration absorption component (134) are disposed symmetrically at the two ends of the length direction of the second connecting member (142).

13. (canceled)

14. The bearing device (100) of claim 6, wherein
the first elastic member (131) includes a first rigid column (1311) and a first elastic element (1312) sleeved on the first rigid column (1311), and the second elastic member (132) includes a second rigid column (1321) and a second elastic element (1322) sleeved on the second rigid column (1321);
the first elastic element (1312) is connected to the support unit (12) through the first rigid column (1311), and two ends of the first elastic element (1312) are in contact with the support unit (12) and the connecting member (14), respectively; and
the second elastic element (1322) is connected to the support unit (12) through the second rigid column (1321), and two ends of the second elastic element (1322) are in contact with the support unit (12) and the connecting member (14), respectively.

15. (canceled)

16. (canceled)

17. The bearing device (100) of claim 6, further comprising a support frame (15) and a support shaft (16), wherein
the connecting member (14) is disposed on two ends of a length direction of the mount (11), and two opposite ends of the support frame (15) are rotatably connected to the connecting member (14) through the support shaft (16).

18. The bearing device (100) of claim 17, wherein
the connecting member (14) is provided with a mounting hole (143) for the support shaft (16) to pass through;
an outer surface of the support shaft (16) is provided with a needle roller component (163); and
the needle roller component (163) is fixed to the connecting member (14) by a lock nut to make the support shaft (16) capable of driving the connecting member (14) to rotate.

19. A mobile digital radiography device (200), comprising an X-ray tube (210), a beam limiter (220), and the bearing device (100) of claim 1, wherein
the X-ray tube (210) is disposed on one side of the mount (11);
a hole (111) is disposed at a position corresponding to the X-ray tube (210) of the mount (11) for a beam of the X-ray tube (210) to pass through; and
the beam limiter (220) disposed on the other side of the mount (11) is configured to receive the beam passing through the hole (111).

20. (canceled)

21. A bearing device (100) of an X-ray tube (210), comprising a bottom plate, a support unit (12), and a vibration absorption unit (13), wherein
the support unit (12) is disposed on the bottom plate and is disposed close to the X-ray tube; and
the vibration absorption unit (13) is disposed on the support unit (12) and includes a first elastic member (131) and a second elastic member (132), a direction where the first elastic member (131) is disposed being parallel to a plane where the support unit (12) is located, an inclination angle being formed between the direction where the first elastic member (131) is disposed and a plane where the bottom plate is located, and a direction where the second elastic member (132) is disposed being perpendicular to the plane where the support unit (12) is located.

22. The bearing device (100) of the X-ray tube (210) of claim 21, wherein
two opposite ends of the first elastic member (131) are installed in the support unit (12); and the inclination angle between the direction where the first elastic member (131) is disposed and the plane where the bottom plate is located is in a range of 44°-46°; or
two opposite ends of the second elastic member (132) are installed in the support unit (12); and one end extends towards the X-ray tube (210) and is spaced apart from the X-ray tube (210).

23. (canceled)

24. The bearing device (100) of the X-ray tube (210) of claim 21, wherein
the support unit (12) includes a first support component (121) and a second support component (122);
the first support component (121) and the second support component (122) are located on a same side of the bottom plate and are fixedly disposed at two opposite ends of the bottom plate, respectively; and
the first support component (121) and the second support component (122) are respectively located at two opposite ends of the X-ray tube (210) and are spaced apart from the X-ray tube (210).

25. The bearing device (100) of the X-ray tube (210) of claim 24, wherein each of the first support component (121) and the second support component (122) is provided with at least one first elastic member (131) and at least one second elastic member (132).

26-30. (canceled)
